(19) United States
(12) Patent Application Publication
(10) Pub. No.: US 2010/0311841 A1
(43) Pub. Date: Dec. 9, 2010

(54) SYNTHETIC SPHINGOLIPID ANALOGS

(76) Inventors: Arie Dagan, Jerusalem (IL); Shimon Gatt, Jerusalem (IL); Shimon Slavin, Tel Aviv (IL)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 12/742,036

(22) PCT Filed: Nov. 6, 2008
(86) PCT No.: PCT/IL08/01459

§ 371 Date: May 7, 2010

(30) Foreign Application Priority Data
Nov. 8, 2007 (IL) 187245

(51) Int. Cl. A61K 31/135; C07C 213/00

(52) U.S. Cl. 514/653; 564/360

(57) ABSTRACT

Therapeutic compounds based on synthetic sphingolipid analogs are provided, particularly alkylthiophenyl substituted ceramide analogs, suitable for treating degenerative, infectious, and other diseases.

### FIELD OF THE INVENTION

The present invention relates to novel therapeutic compounds, particularly suitable for treating metabolic diseases, degenerative and proliferative disorders, and infectious diseases.

### BACKGROUND OF THE INVENTION

During the past decade there has been an enormous increase in research on sphingolipids due to discoveries that implicated members of this group in signal transduction processes [recently reviewed in Fernandis et al., Curr. Opin. Lipidol. 18: 121-8, (2007); Levade et al., Biochim. Biophys. Acta 1438, 1-17 (1999); Mathias et al., Biochem. J. 335, 465-480 (1998); Perry et al., Biochim. Biophys. Acta 1436, 233-243 (1998); Riboni et al., Prog. Lipid Res. 36, 153-195 (1997)]. The most studied compound was ceramide which was shown to play a role in the regulation of key processes such as growth inhibition, differentiation and apoptosis [Hannun et al., Biochim. Biophys. Acta 1154, 223-36; Hannun et al., Trends Cell Biol. 10, 73-80 (2001); Higgins et al., Trends Biochem. Sci. 17, 18-21 (1992)].

The role of sphingolipids in signal transduction [reviewed in Eyster K M Adv Physiol Educ. 31: 5-16, (2007); L. Zheng et al Biochim Biophys Acta. 1758: 1864-84, (2006); Riboni et al., Prog. Lipid Res. 36, 153-195 (1997); and A. Gomez-Munoz, Biochim. Biophys. Acta 1391, 32-109 (1998)] have been extensively studied, and was proposed to operate through the “sphingomyelin cycle”. According to this hypothesis, binding a particular extracellular ligand to its receptor activates a plasma membrane-bound sphingomyelinase, giving rise to ceramide, which acts as a mediator of the intracellular effects of the ligand. Numerous publications describe and emphasize the role of ceramide in cell killing by apoptosis as well as its effect on important cellular events such as proliferation, differentiation and reaction to stress conditions [Morales et al., Mini Rev. Med. Chem. 7: 371-82 (2007)]. Of particular interest are also reports that short-chain cell-permeable (e.g., C₂ or C₆) ceramides evoke biological responses that lead to cell killing. Other studies, using the precursor of ceramide—sphingosine—have shown its effects on cell growth and viability. Furthermore, sphingosine was shown to inhibit protein kinase C and increase the intracellular concentration of calcium ions. The phosphorylated form of sphingosine, i.e., sphingosine-1-phosphate has been shown to be a potent activator of phospholipase D; di- or tri-methylated sphingosine was shown to inhibit growth of cancer cells [Endo et al., Cancer Research 51, 1613-8, (1981)].

WO 03/027058 relates to a group of compounds suitable for the treatment of parasitic diseases and cancerous diseases for killing of wild type and drug-resistant cancer cells, particularly by inhibiting the synthesis of sphingolipids and ceramides. The compounds disclosed in WO 03/027058 essentially have an alkyl backbone substituted with an alkyl or alkenyl chain which itself may be substituted.

It has now been found that the compounds of WO 03/027058 are also effective against immuno-degenerative disorders, in particular against GVHD (Graft Versus Host Disease). GVHD is a type of incompatibility reaction of transplanted cells against host tissues that possess an antigen not possessed by the donor; it is a common complication of allogeneic bone marrow transplantation. After bone marrow transplantation, T cells present in the graft, either as contaminants or intentionally introduced into the host, attack the tissues of the transplant recipient after perceiving host tissues as antigenically foreign. A wide range of host antigens can initiate graft-versus-host-disease, among them the HLAs. However, graft-versus-host disease can occur even when HLA-identical siblings are the donors. HLA-identical siblings or HLA-identical unrelated donors (called a minor mismatch as opposed to differences in the HLA antigens, which constitute a major mismatch) often still have genetically different proteins that can be presented on the MHC.

While donor T-cells are undesirable as effector cells of GVHD, they are valuable for engraftment by preventing the recipient's residual immune system from rejecting the bone marrow graft (host-versus-graft). Additionally, as bone marrow transplantation is frequently used to cure malignant disorders (most prominently the leukemias), donor T-cells have proven to have a valuable graft-versus-tumor effect. A great deal of current research on allogeneic bone marrow transplantation involves attempts to separate the undesirable GVHD aspects of T-cell physiology from the desirable graft-versus-tumor effect.

Clinically, GVHD is divided into acute and chronic forms. The acute or fulminant form of the disease is observed within the first 100 days post-transplant, and the chronic form of GVHD is defined as that which occurs after 100 days. This distinction is not arbitrary; acute and chronic GVHD appear to involve different immune cell subsets, different cytokine profiles, and different types of target organ damage.

Classically, acute GVHD is characterized by selective damage to the liver, skin and mucosa, and the gastrointestinal tract. Newer research indicates that other GVHD target organs include the immune system (the hematopoietic system, e.g. the bone marrow and the thymus) itself, and the lungs in the form of idiopathic pneumonitis. Chronic GVHD damages the above organs, but also causes changes to the connective tissue (e.g. of the skin and exocrine glands).

GVHD can largely be avoided by performing a T-cell depleted bone marrow transplant. These types of transplants result in reduced target organ damage and generally less GVHD, but at a cost of diminished graft-versus-tumor effect, a greater risk of engraftment failure, and general immunodeficiency, resulting in a patient more susceptible to viral, bacterial, and fungal infection. Methotrexate and cyclosporin are common drugs used for GVHD prophylaxis. In a multi-center study [Lancet 2005 Aug. 27-Sep. 2; 366(9487):733-41], disease-free survival at 3 years was not different between T cell depleted and T cell replete transplants.

It is an object of the present invention to provide novel compounds which are in particular suitable for treating anti-proliferative disorders.

It is another object of the present invention to provide novel compounds and combination of compounds which are suitable in treating immuno-degenerative disorders, in particular GVHD.

It is a further object of the present invention to provide novel sphingolipid analogs.

It is a still further object of the present invention to provide novel sphingolipid analogs with a potential for treating neurodegenerative disorders, metabolism-associated conditions, and infectious diseases.

### SUMMARY OF THE INVENTION

The invention provides a compound of formula (I):

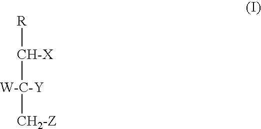

wherein
R represents a substituent selected from

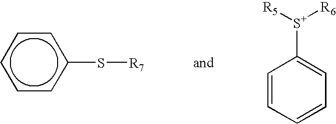

wherein R₇ represents C₁₋₆alkyl or C₁₋₆alkenyl; R₅ and R₆ independently represent C₁₋₆ alkyl or C₁₋₆alkenyl; or alternatively R₅ represents C₇₋₂₄ alkyl or alkenyl, and R₆ independently represents C₁₋₆ alkyl or C₁₋₆ alkenyl group or hydrogen; X represents hydrogen or the group —OR₄ in which R₄ is hydrogen or a linear or branched C₁-C₆ alkyl or alkenyl chain which may be optionally substituted with hydroxyl; Y represents —NHR^{x} wherein R^{x} is hydrogen, a linear or branched alkyl or alkenyl chain which may be optionally substituted with hydroxyl, or an amino protecting group;

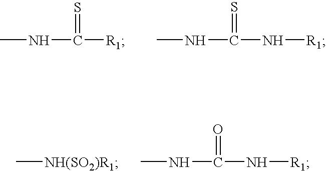

wherein R₁ in four preceding formulae represents C₁₋₆ alkyl or C₁₋₆alkenyl; —NR₁R₂ wherein R₁ and R₂ independently represent C₁₋₆ alkyl or C₁₋₆alkenyl, or R₁ represents C₇₋₂₄ alkyl or alkenyl while R₂ independently represents C₁₋₆ alkyl or C₁₋₆ alkenyl group or hydrogen; —N⁺R₁R₂R₃, wherein R₁, R₂ and R₃ independently represent C₁₋₆alkyl or C₁₋₆alkenyl, or R₁ represents C₇₋₂₄ alkyl or alkenyl while R₂ and R₃ independently represent C₁₋₆ alkyl or C₁₋₆ alkenyl group or hydrogen;

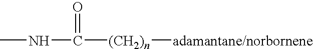

wherein n is zero or an integer of from 1 to 20; —NH adamantane/norbornene;

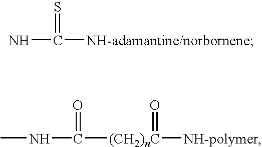

where “polymer” designates a natural or synthetic biocompatible polymer having a molecular weight between 10³ and 10⁶ daltons; W represents hydrogen or —CH₂—O—R₈, wherein R₈ is hydrogen or a linear or branched C₁-C₆ alkyl or alkenyl chain which may be optionally substituted with hydroxyl; and Z represents hydrogen, —OH, a monosaccharide or disaccharide, a monosaccharide sulfate, or choline phosphate; and isomers and pharmaceutically acceptable salts thereof.

In a preferred embodiment of the invention Y in the compound of formula (I) is NHR^{x} wherein R^{x} has the meaning as described above. In another preferred compound of formula (I) Z is —OH and/or X is OH. In a preferred embodiment of the invention, W in a compound having formula (I) is H. Said amino protecting group may be selected, for example, from tBOC, FMOC and CBZ. In preferred compounds according to the invention substituent R in formula (I) is 4-alkylthiophenyl, particularly 4-methylthiophenyl. On other preferred embodiment, said R is. 4-dimethylthiophenyl.

The invention further provides a compound having the formula (designated AD2813):

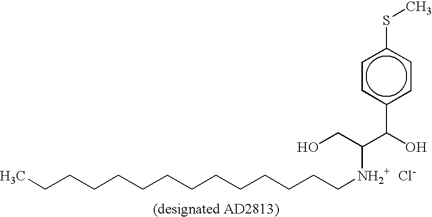

The invention relates to a pharmaceutical composition comprising as active ingredient a compound of formula (I)

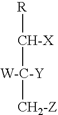

wherein the symbols have the same meaning as defined above, or isomers and pharmaceutically acceptable salts thereof, and further optionally pharmaceutically acceptable carrier, adjuvant or diluent. In a preferred embodiment of the invention, said composition comprises a compound of formula (I) in which Y is NHR^{x}, wherein R^{x} has the same meaning as described above. In a preferred composition of the invention Z in formula (I) is —OH and/or X is OH and/or W is H. A preferred composition of the invention comprises compound AD2813:

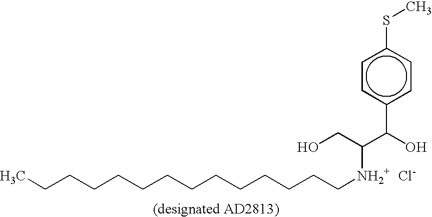

The invention provides pharmaceutical compositions for the treatment of a disorder selected from the group consisting of proliferative disorders, neurodegenerative disorders, metabolism-associated conditions, infectious diseases, and immunity-associated conditions. Said proliferative disorder is particularly a cancerous growth, for example prostate or bladder cancer. Said neurode generative disorder is in a preferred embodiment Alzheimer's disease. Said metabolism-associated condition is selected from diabetes, cystic fibrosis, and lipid storage diseases. Said infectious disease is selected from the group consisting of viral infections, bacterial infections, fungal infections, and protozoal infections. It is understood that parasitic diseases are included among said infectious diseases. In a preferred embodiment of the invention, said immunity-associated condition is GVHD or allergy. Said infectious disease is preferably selected from mycoplasma infection, leishmaniasis, and malaria. In case of cancerous growth, said pharmaceutical composition is preferably employed for killing of wild type and drug-resistant cancer cells, especially prostate and bladder carcinoma. Said pharmaceutical composition is further preferably employed for the selective killing of drug-resistant cancer cells.

The invention is directed to a method of treating a disorder selected from the group consisting of proliferative disorders, neurodegenerative disorders, metabolism-associated conditions, infectious diseases, and immunity-associated conditions in a patient in need of such treatment, comprising administering to said patient a therapeutically effective amount of a compound of formula (I) as defined above, or of an isomer or a pharmaceutically acceptable salt thereof. Said substituent Y is preferably NHR^{x}, wherein R^{x} has the same meaning as described, said substituent Z is preferably OH, said X is preferably OH, and said W is preferably H. A preferred compound to be administered in said method is AD2813:

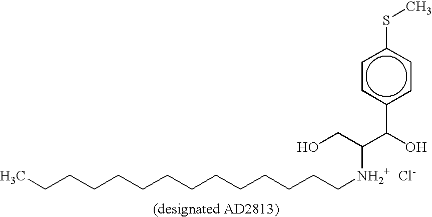

The invention further relates to the use of compounds described above in the preparation of a medicament for treating a disorder selected from the group consisting of proliferative disorders, neurodegenerative disorders, metabolism-associated conditions, infectious diseases, and immunity-associated conditions.

### BRIEF DESCRIPTION OF THE DRAWINGS

The above and other characteristics and advantages of the invention will be more readily apparent through the following example, and with reference to the appended drawing, wherein:

FIG. 1. shows the treatment of whole-body-irradiated mice with a compound according to the invention, AD-2813.

### DETAILED DESCRIPTION OF THE INVENTION

New sphingolipid analogs have now been synthesized, exhibiting surprisingly strong antiproliferative effects. Said compounds are potent in killing a variety of cells, including drug-sensitive and drug-resistant cells, alone or in combination with other anti-cancer drugs. The pharmaceutical compositions comprising them are thus particularly intended for the treatment of cell proliferative, especially cancerous, diseases. Sphingolipid analogs of the invention are further useful for treating cystic fibrosis, Alzheimer disease, leishmaniasis, mycoplasma infections, bacterial infections, fungal infections, viral infections, allergy, diabetes, malaria, lipid storage diseases, such as Gaucher, Nieman-Pick, Fabry, Farber and Tay-Sachs disease. The pharmaceutical compositions of the invention are further intended for the treatment of immuno-degenerative diseases, in particular GVHD (Graft Versus Host Disease).

Sphingolipid analogs of the following formula (I) are provided:

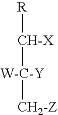

wherein
R represents a substituent selected from

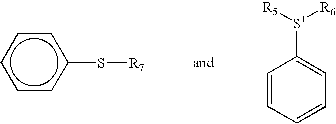

wherein R₇ represents C₁₋₆alkyl or C₁₋₆alkenyl;
R₅ and R₆ independently represent C₁₋₆ alkyl or C₁₋₆alkenyl; or alternatively R₅ represents C₇₋₂₄ alkyl or alkenyl, and R₆ independently represents C₁₋₆ alkyl or C₁₋₆ alkenyl group or hydrogen; X represents hydrogen or the group —OR₄ in which R₄ is hydrogen or a linear or branched C₁-C₆ alkyl or alkenyl chain which may be optionally substituted with hydroxyl; Y represents —NHR^{x} wherein R^{x} is hydrogen, a linear or branched alkyl or alkenyl chain which may be optionally substituted with hydroxyl, or an amino protecting group;

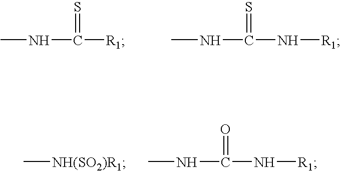

wherein R₁ in four preceding formulae represents C₁₋₆ alkyl or C₁₋₆alkenyl; —NR₁R₂ wherein R₁ and R₂ independently represent C₁₋₆ alkyl or C₁₋₆alkenyl, or R₁ represents C₇₋₂₄ alkyl or alkenyl while R₂ independently represents C₁₋₆ alkyl or C₁₋₆ alkenyl group or hydrogen; —N⁺R₁R₂R₃, wherein R₁, R₂ and R₃ independently represent C₁₋₆alkyl or C₁₋₆alkenyl, or R₁ represents C₇₋₂₄ alkyl or alkenyl while R₂ and R₃ independently represent C₁₋₆ alkyl or C₁₋₆ alkenyl group or hydrogen;

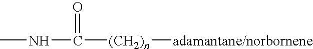

wherein n is zero or an integer of from 1 to 20; —NH-adamantane/norbornene;

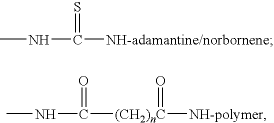

where “polymer” designates a natural or synthetic biocompatible polymer having a molecular weight between 10³ and 10⁶ daltons; W represents hydrogen or —CH₂—O—R₈, wherein R₈ is hydrogen or a linear or branched C₁-C₆ alkyl or alkenyl chain which may be optionally substituted with hydroxyl; and Z represents hydrogen, —OH, a monosaccharide or disaccharide, a monosaccharide sulfate, or choline phosphate; and isomers and pharmaceutically acceptable salts thereof.

Said R is preferably 4-methylthiophenyl. Said X may be OH. Said W is preferably H. Said Y is preferably —NHR^{x} wherein R^{x} is an alkyl, for example linear C₁₀₋₂₆ alkyl, such as C₁₄alkyl.

Methylthiophenyl analogs of sphingolipids, as well as their isomers and pharmaceutically acceptable salts, are suitable for using in the preparation of medicaments for treating proliferative disorders, neurodegenerative disorders, metabolism-associated conditions, infectious diseases, and immunity-associated conditions.

In one particular embodiment, the present invention relates to a compound of formula (I), the compound being

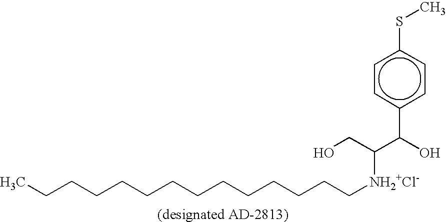

Compound AD-2813 turned out to have a remarkable potency for curing human tumors in a nude mouse model. Of course, other salt than chloride may be used.

The invention also relates to a pharmaceutical composition comprising as active ingredient a compound of formula (I) wherein the substituents are as defined above, and optionally further comprising pharmaceutically acceptable carrier, adjuvant or diluent. In one particular embodiment, the present invention relates to a pharmaceutical composition comprising as an active ingredient the compound of formula (I) being AD-2813.

In a further aspect the invention relates to a method of treating a cell proliferative, particularly cancerous disease, specifically for killing of wild type and drug-resistant cancer cells in a patient in need of such treatment, comprising administering to said patient a therapeutically effective amount of a compound of formula (I) or of pharmaceutical composition comprising the same. In one particular embodiment, the present invention relates to a method of treating a cancerous disease, particularly for killing of wild type and drug-resistant cancer cells, in a patient in need of such treatment comprising administering to said patient a therapeutically effective amount of said compound AD-2813.

In a preferred embodiment, a pharmaceutical compositions comprising at least one compound of above formula (I) is used for the treatment of immuno-degenerative disorders, particularly GVHD.

The compounds of the invention are generally provided in the form of pharmaceutical compositions. Said compositions are for use by injection or by oral uptake.

The pharmaceutical compositions of the invention generally comprise a buffering agent, an agent which adjusts the osmolarity thereof, and optionally one or more carriers, excipients and/or additives as known in the art, e.g., for the purposes of adding flavors, colors, lubrication, or the like to the pharmaceutical composition.

Each carrier should be both pharmaceutically and physiologically acceptable in the sense of being compatible with the other ingredients and not injurious to the subject to be treated. While formulations include those suitable for rectal, nasal, preferred formulations are intended for oral or parenteral administration, including intramuscular, intradermal, subcutaneous and specifically intravenous administration. The formulations may conveniently be presented in unit dosage form and may be prepared by any methods known in the art of pharmacy.

Carriers may include starch and derivatives thereof, cellulose and derivatives thereof, e.g., microcrystalline cellulose, xantham gum, and the like. Lubricants may include hydrogenated castor oil and the like.

As used herein “pharmaceutically acceptable carrier” includes any and all solvents, dispersion media, and coatings, not harmful to the subject. Antibacterial and antifungal agents may be included. The use of such media and agents for pharmaceutical active substances is well known in the art.

A preferred pharmaceutical formulation is preferably used for administration by injection, including intravenous injection.

The compositions of the invention may be administered in a variety of ways. By way of non-limiting example, the composition may be delivered by injection intravenously, intramuscularly, or intraperitoneally. Intravenous administration, for example, is advantageous.

The pharmaceutical forms suitable for injection use include sterile aqueous solutions or dispersions and sterile powders for the extemporaneous preparation of sterile injectable solutions or dispersions. In all cases the form must be sterile and must be fluid to the extent that easy syringeability exists. It must be stable under the conditions of manufacture and storage and must be preserved against the contaminating action of microorganisms, such as bacteria and fungi. The carrier can be solvent or dispersion medium containing, for example, water, ethanol, polyol (for example, glycerol, propylene glycol, and liquid polyethylene glycol, and the like), suitable mixtures thereof, and vegetable oils. The proper consistency can be maintained, for example, by the use of a coating, such as lecithin, by the maintenance of the required particle size in the case of dispersion, and by the use of surfactants.

Sterile injectable solutions are prepared by incorporating the active compounds in the required amount in the appropriate solvent with various of the other ingredients enumerated above, as required, followed by filter sterilization. Generally, dispersions are prepared by incorporating the various sterilized active ingredients into a sterile vehicle which contains the basic dispersion medium and the required other ingredients from those enumerated above.

In the case of sterile powders for the preparation of the sterile injectable solutions, the preferred method of preparation are vacuum-drying and freeze drying techniques which yield a powder of the active ingredient plus any additional desired ingredient from a previously sterile-filtered solution thereof.

The preparation of pharmaceutical compositions is well known in the art and has been described in many articles and textbooks, see e.g., Remington's Pharmaceutical Sciences, Gennaro A. R. ed., Mack Publishing Company, Easton, Pa., 1990, and especially pages 1521-1712 therein. Additives may also be designed to enhance uptake of the active agent across cell membranes. Such agents are generally agents that will enhance cellular uptake of the molecules of the invention. For example, the compounds of the invention may be enclosed within liposomes. The preparation and use of liposomes, e.g., using particular transfection reagents, is well known in the art. Other methods of obtaining liposomes include the use of Sendai virus or of other viruses.

The dose of the active agent may vary. The dose would generally depend on the disease, the state of the disease, age, weight and sex of the patient, and is to be determined by the attending physician.

A number of methods of the art of molecular biology are not detailed herein, as they are well known to the person of skill in the art. Textbooks describing such methods are e.g., Sambrook et al., Molecular Cloning, A Laboratory Manual, Cold Spring Harbor Laboratory, ISBN: 0879693096, 1989; Current Protocols in Molecular Biology by F. M. Ausubel, ISBN: 047150338X, John Wiley & Sons, Inc. 1988; and Short Protocols in Molecular Biology by F. M. Ausubel et al. (eds.) 3rd ed., John Wiley & Sons, ISBN: 0471137812, 1995. Furthermore, a number of immunological techniques are not in each instance described herein in detail, as they are well known to the person of skill in the art (see, for example, Current Protocols in Immunology, Coligan et al. (eds), John Wiley & Sons. Inc., New York, N.Y.).

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Disclosed and described, it is to be understood that this invention is not limited to the particular examples, process steps, and materials disclosed herein as such process steps and materials may vary somewhat. It is also to be understood that the terminology used herein is used for the purpose of describing particular embodiments only and not intended to be limiting since the scope of the present invention will be limited only by the appended claims and equivalents thereof.

The following examples are thus only representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

### Example 1

### Mice Experiment Using AD-2813

BALB/NUDE mice were whole body irradiated with 400 cGy, 10 days later they were injected s.c. with 3.5*10e6 TSU-PR1 cells/mouse which are commonly considered as prostate carcinoma cells but recently are also considered by some as bladder carcinoma cells. Eight days later, tumors were formed, and mice were separated into 3 groups:

Group 1: a control without any injection.

Group 2: were injected s.c. with vehicle (cremophor)

Group 3: were treated s.c. daily with AD-2813 (20 mg/kg in cremophor) close to tumor.

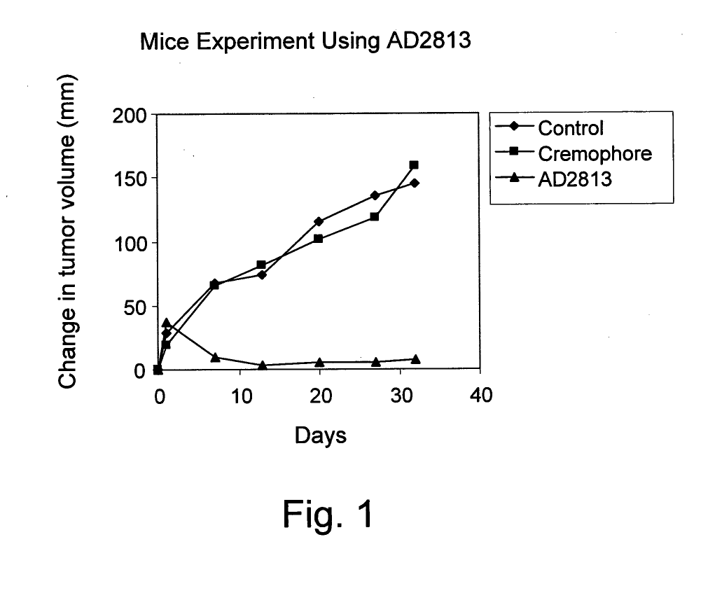

The results, as presented in FIG. 1, show the significant decrease in tumor volume after about 10 days of treatment with AD-2813. The drastic change in tumor volume is pronounced particularly when compared to the control and treatment with cremophor groups.

While the invention has been described using some specific examples, many modifications and variations are possible. It is therefore understood that the invention is not intended to be limited in any way, other than by the scope of the appended claims.

1. A compound of formula (I): 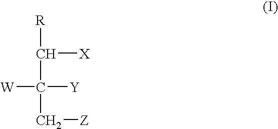
wherein
R represents a substituent selected from 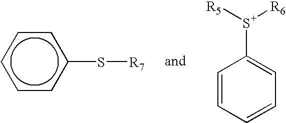
wherein R₇ represents C₁₋₆alkyl or C₁₋₆alkenyl;
R₅ and R₆ independently represent C₁₋₆ alkyl or C₁₋₆alkenyl; or alternatively R₅ represents C₇₋₂₄ alkyl or alkenyl, and R₆ independently represents C₁₋₆ alkyl or C₁₋₆ alkenyl group or hydrogen;
X represents hydrogen or the group —OR₄ in which R₄ is hydrogen or a linear or branched C₁-C₆ alkyl or alkenyl chain which may be optionally substituted with hydroxyl;
Y represents —NHR^{x} wherein R^{x} is hydrogen, a linear or branched alkyl or alkenyl chain which may be optionally substituted with hydroxyl, or an amino protecting group; 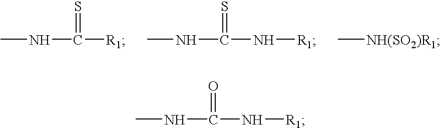
wherein R₁ in four preceding formulae represents
C₁₋₆ alkyl or C₁₋₆ alkenyl; —NR₁R₂ wherein R₁ and R₂ independently represent C₁₋₆ alkyl or C₁₋₆ alkenyl, or R₁ represents C₇₋₂₄ alkyl or alkenyl while R₂ independently represents C₁₋₆ alkyl or C₁₋₆ alkenyl group or hydrogen; —N⁺R₁R₂R₃, wherein R₁, R₂ and R₃ independently represent C₁₋₆alkyl or C₁₋₆alkenyl, or R₁ represents C₇₋₂₄ alkyl or alkenyl while R₂ and R₃ independently represent C₁₋₆ alkyl or C₁₋₆ alkenyl group or hydrogen; 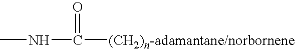
wherein n is zero or an integer of from 1 to 20; —NH-adamantane/norbornene; 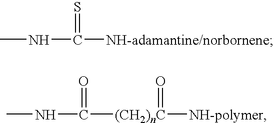
where “polymer” designates a natural or synthetic biocompatible polymer having a molecular weight between 10³ and 10⁶ daltons;
W represents hydrogen or —CH₂—O—R₈, wherein R₈ is hydrogen or a linear or branched C₁-C₆ alkyl or alkenyl chain which may be optionally substituted with hydroxyl; and
Z represents hydrogen, —OH, a monosaccharide or disaccharide, a monosaccharide sulfate, or choline phosphate;
and isomers and pharmaceutically acceptable salts thereof.

2. A compound according to claim 1, wherein Y is NHR^{x}, wherein R^{x} is selected from hydrogen and a linear or branched alkyl or alkenyl chain which may be optionally substituted with hydroxyl.

3. A compound according to claims 1 wherein Z is —OH.

4. A compound according to claim 1 wherein X is OH.

5. A compound according to claim 1 wherein. W is H.

6. A compound according to claim 1 wherein R is 4-alkylthiophenyl.

7. A compound according to claim 1 wherein R is 4-methylthiophenyl or 4-dimethylthiophenyl.

8. (canceled)

9. (canceled)

10. A compound according to claim 1, being the compound

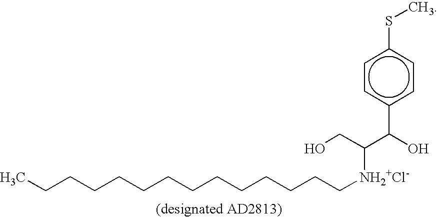

11. A pharmaceutical composition comprising as active ingredient a compound of formula (I) 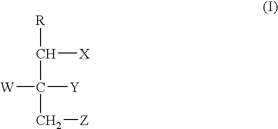
wherein
R represents a substituent selected from 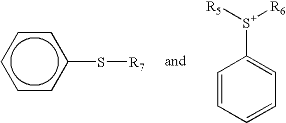
wherein R₇ represents C₁₋₆alkyl or C₁₋₆alkenyl;
R₅ and R₆ independently represent C₁₋₆ alkyl or C₁₋₆alkenyl; or alternatively R₅ represents C₇₋₂₄ alkyl or alkenyl, and R₆ independently represents C₁₋₆ alkyl or C₁₋₆ alkenyl group or hydrogen;
X represents hydrogen or the group —OR₄ in which R₄ is hydrogen or a linear or branched C₁-C₆ alkyl or alkenyl chain which may be optionally substituted with hydroxyl;
Y represents —NHR^{x} wherein R^{x} is hydrogen, a linear or branched alkyl or alkenyl chain which may be optionally substituted with hydroxyl, or an amino protecting group; 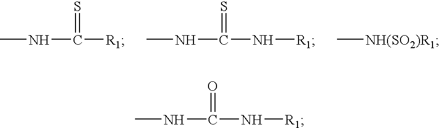
wherein R₁ in four preceding formulae represents C₁₋₆ alkyl or C₁₋₆alkenyl; —NR₁R₂ wherein R₁ and R₂ independently represent C₁₋₆ alkyl or C₁₋₆alkenyl, or R₁ represents C₇₋₂₄ alkyl or alkenyl while R₂ independently represents C₁₋₆ alkyl or C₁₋₆ alkenyl group or hydrogen; —N⁺R₁R₂R₃, wherein R₁, R₂ and R₃ independently represent C₁₋₆alkyl or C₁₋₆alkenyl, or R₁ represents C₇₋₂₄ alkyl or alkenyl while R₂ and R₃ independently represent C₁₋₆ alkyl or C₁₋₆ alkenyl group or hydrogen; — 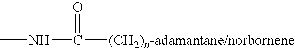
wherein n is zero or an integer of from 1 to 20; —NH-adamantane/norbornene; 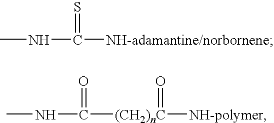
where “polymer” designates a natural or synthetic biocompatible polymer having a molecular weight between 10³ and 10⁶ daltons;
W represents hydrogen or —CH₂—O—R₈, wherein R₈ is hydrogen or a linear or branched C₁-C₆ alkyl or alkenyl chain which may be optionally substituted with hydroxyl; and
Z represents hydrogen, —OH, a monosaccharide or disaccharide, a monosaccharide sulfate, or choline phosphate;
and isomers and pharmaceutically acceptable salts thereof;
and optionally further comprising pharmaceutically acceptable carrier, adjuvant or diluent.

12. (canceled)

13. (canceled)

14. (canceled)

15. (canceled)

16. A composition according to claim 11, wherein said compound of formula (I) is compound AD2813:

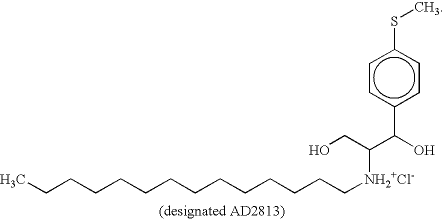

17. A pharmaceutical composition according to claim 11, for the treatment of a disorder selected from the group consisting of proliferative disorders, neurodegenerative disorders, metabolism-associated conditions, infectious diseases, and immunity-associated conditions.

18. A pharmaceutical composition according to claim 17 wherein said proliferative disorder is a cancerous growth.

19. (canceled)

20. (canceled)

21. (canceled)

22. A pharmaceutical composition according to claim 17 wherein said immunity-associated condition is GVHD or allergy.

23. (canceled)

24. (canceled)

25. (canceled)

26. A method of treating a disorder selected from the group consisting of proliferative disorders, neurodegenerative disorders, metabolism-associated conditions, infectious diseases, and immunity-associated conditions in a patient in need of such treatment, comprising administering to said patient a therapeutically effective amount of a compound of formula (I) as defined in claim 1, or of an isomer or a pharmaceutically acceptable salt thereof.

27. A method according to claim 26, being the compound

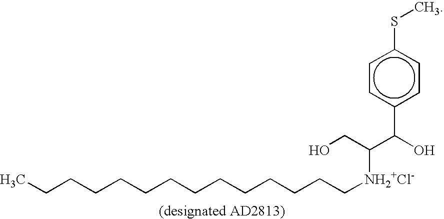

28. (canceled)
